(19) United States
(12) Patent Application Publication
(10) Pub. No.: US 2013/0089611 A1
(43) Pub. Date: Apr. 11, 2013

(54) RASAGILINE CITRAMIDE

(71) Applicant: TEVA PHARMACEUTICAL INDUSTRIES, LTD., Petach-Tikav (IL)

(72) Inventors: Konstantin Ulanenko, Netanya (IL); Gregory Verba, Haifa (IL); Muhammad Safadi, Nazareth (IL); Anton Frenkel, Netanya (IL); Michal Keisar, Kfar-Yona (IL); Danit Licht, Givat Shmuel (IL); Eliezer Bahar, Tel Aviv (IL); Ramy Lidor-Hadas, Kfar-Saba (IL)

(73) Assignee: TEVA PHARMACEUTICAL INDUSTRIES, LTD., Petach-Tikva (IL)

(21) Appl. No.: 13/647,658

(22) Filed: Oct. 9, 2012

Related U.S. Application Data
(60) Provisional application No. 61/545,414, filed on Oct. 10, 2011.

(51) Int. Cl. A61K 31/197; A61P 25/16; A61K 9/28; A61K 9/36; C07C 235/74; C07C 231/14

(52) U.S. Cl. 424/479; 562/457; 514/563; 424/474

(57) ABSTRACT

The subject invention provides rasagiline citramide and a composition containing N-propargyl-1(R)-aminoindan or a pharmaceutically acceptable salt thereof, and a compound of rasagiline citramide or a salt thereof.

This application claims benefit of U.S. Provsional Application No. 61/545,414, filed Oct. 10, 2011, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

U.S. Pat. Nos. 5,532,415, 5,387,612, 5,453,446, 5,457,133, 5,599,991, 5,744,500, 5,891,923, 5,668,181, 5,576,353, 5,519,061, 5,786,390, 6,316,504, 6,630,514, 7,750,051, and 7,855,233 disclose R(+)-N-propargyl-1-aminoindan (“R-PAI”), also known as rasagiline, and its pharmaceutically acceptable salts. These U.S. patents also disclose that rasagiline is a selective inhibitor of the B-form of the enzyme monoamine oxidase (“MAO-B”) and is useful in treating Parkinson's disease and various other conditions by inhibition of MAO-B in the brain.

U.S. Pat. Nos. 6,126,968, 7,572,834, and 7,598,420, U.S. patent application Ser. Nos. 12/283,022, and 12/283,107 and PCT publications WO 95/11016 and WO 2006/014973, hereby incorporated by reference, disclose pharmaceutical compositions comprising rasagiline and processes for their preparation.

AZILECT® is a commercially available rasagiline mesylate immediate release formulation indicated for the treatment of the signs and symptoms of idiopathic Parkinson's disease as initial monotherapy and as adjunct therapy to levodopa. The current marketed formulation of rasagiline (Azilect®) is rapidly absorbed, reaching peak plasma concentration (tₘₐₓ) in approximately 1 hour. The absolute bioavailability of rasagiline is about 36%. (AZILECT® Product Label, May 2006)

### SUMMARY OF THE INVENTION

The subject invention provides an isolated compound having the structure:

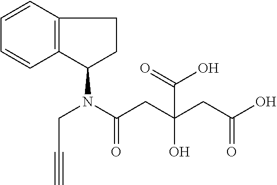 [SMILES: C#CCN(C(=O)CC(O)(CC(=O)O)C(=O)O)[C@@H]1CCc2ccccc21]

or a salt thereof.

The subject invention also provides a composition comprising a compound having the structure:

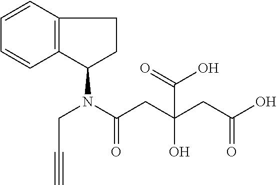 [SMILES: C#CCN(C(=O)CC(O)(CC(=O)O)C(=O)O)[C@@H]1CCc2ccccc21]

or a salt thereof, wherein the composition is free of rasagiline or a salt thereof.

The subject invention further provides a process for preparing rasagiline citramide comprising the steps of:
a) mixing citric acid with thionyl chloride in a first solvent under an inert atmosphere at a temperature of less than 30° C. to obtain trimethyl citrate;
b) mixing trimethyl citrate obtained from step a) and NaOH solution in a second solvent at a temperature of less than 30° C. to obtain 1,2-dimethyl citrate;
c) mixing 1,2-Dimethyl citrate obtained from step b) and thionyl chloride in a third solvent at a temperature of less than 30° C. to obtain a residue oil;
d) mixing the residue oil from step c) and a mixture of rasagilne and triethylamine in the third solvent at a temperature of less than 30° C. to obtain 1-rasagiline-2,3-dimethyl citramide; and
e) mixing an aqueous solution of LiOH and 1-rasagiline-2,3-dimethyl citramide obtained from step d) in a combination of solvents at a temperature of less than 30° C.; and
f) adjusting the pH of the reaction mixture of step e) with an acid to obtain 1-rasagiline citramide.

The subject application yet further provides a pharmaceutical composition comprising rasagiline or a pharmaceutically acceptable salt thereof, citric acid, rasagiline citramide or a salt thereof, and at least one pharmaceutically acceptable carrier, wherein rasagiline citramide is present in the pharmaceutical composition in an amount greater than about 0.03%, by weight, relative to the amount of rasagiline, based on a determination by an HPLC method, and wherein if the pharmaceutical composition is at least six months old then the temperature of the pharmaceutical composition during such period did not exceed ambient temperature for a total period of four months or more.

The subject invention yet further provides the pharmaceutical composition disclosed herein in tablet form.

The subject invention yet further provides a pharmaceutical composition in tablet form comprising a core and a coating, wherein the core of the tablet comprises an amount of rasagiline or a pharmaceutically acceptable salt thereof, citric acid and mannitol, wherein the weight ratio of mannitol to citric acid is between 45 to 1 and 10 to 1, and further comprises rasagiline citramide or a salt thereof such that the rasagiline citramide is present in the pharmaceutical composition in an amount greater than about 0.03%, by weight, relative to the amount of rasagiline, based on a determination by an HPLC method.

The invention yet further provides a process for preparing a pharmaceutical composition comprising rasagiline or a pharmaceutically acceptable salt thereof, and at least one pharmaceutically acceptable carrier, comprising:
a) obtaining a batch of rasagiline or a pharmaceutically acceptable salt thereof;
b) determining the amount of rasagiline citramide in the batch using a suitable apparatus; and
c) preparing the pharmaceutical composition from the batch only if the batch is determined to have less than about 1.0% rasagiline citramide by weight relative to the amount of rasagiline.

The invention yet further provides a process for preparing a packaged pharmaceutical composition comprising rasagiline or a pharmaceutically acceptable salt thereof comprising:
a) obtaining a pharmaceutical composition of rasagiline or a pharmaceutically acceptable salt thereof;
b) analyzing the pharmaceutical composition for the presence of rasagiline citramide by a suitable apparatus; and
c) packaging the pharmaceutical composition only if the amount of rasagiline citramide is not more than about 1.0% by weight relative to the amount of rasagiline.

The invention yet further provides a process of distributing a validated batch of a pharmaceutical composition comprising rasagiline or a pharmaceutically acceptable salt thereof and at least one pharmaceutically acceptable carrier, comprising:
a) obtaining a batch of the pharmaceutical composition;
b) performing stability testing with a sample of the batch;
c) determining the total amount of rasagiline citramide in the sample of the batch by a suitable apparatus after stability testing;
d) validating the batch for distribution only if the sample of the batch after stability testing is determined to have not more than about 1.0% by weight of rasagiline citramide relative to the amount of rasagiline; and
e) distributing the validated batch.

The invention yet further provides rasagiline citramide or a salt thereof for use, as a reference standard to detect trace amounts of rasagiline citramide in a pharmaceutical composition comprising rasagiline or a pharmaceutically acceptable salt of rasagiline.

The invention yet further provides a method for treating Parkinson's disease in a patient comprising administering to the patient an amount of the pharmaceutical compositions disclosed herein effective to treat Parkinson's disease in the patient.

### DETAILED DESCRIPTION OF THE INVENTION

R(+)-N-propargyl-1-aminoindan (“R-PAI”), so known as rasagiline, is a small molecule having the following chemical structure:

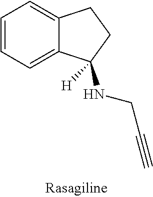

Rasagiline has been reported to be a selective inhibitor of the B-form of the enzyme monoamine oxidase (“MAO-B”) and is useful in treating Parkinson's disease and various other conditions by inhibition of MAO-B in the brain.

A pharmaceutically acceptable salt of rasagiline, rasagiline citrate, and the process of preparing the same has been described in U.S. Pat. No. 7,855,233, the entire content of which is hereby incorporated by reference.

Crystalline rasagiline, and the process of preparing the same has been described in U.S. Pat. Nos. 7,750,051 and 7,968,749, the entire contents of which are hereby incorporated by reference.

Delayed release rasagiline formulations have been described in United States Application Publication Nos. 2009/0181086, 2010/0189790, 2010/0189788, 2010/0189787, and 2010/0189791, the entire content of each of which is hereby incorporated by reference.

It has been found that when rasagiline drug product is exposed to accelerated conditions, an impurity is formed. This impurity was identified to be rasagiline citramide, having the following structure:

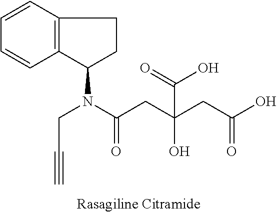 [SMILES: C#CCN(C(=O)CC(O)(CC(=O)O)C(=O)O)[C@@H]1CCc2ccccc21]

Not to be bound by any particular theory, this impurity can be formed via a reaction between rasagiline base and citric acid at elevated temperature during preparation of rasagiline composition or after a prolonged period of storage of rasagiline drug product at accelerated storage conditions.

Other impurities in rasagiline formulations should be avoided, such as R(+)-N-methyl-propargyl-aminoindan and R(+)-N-formyl-propargyl-aminoindan.

The subject invention provides an isolated compound having the structure:

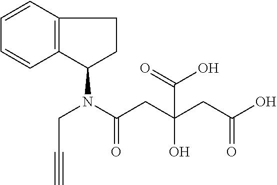 [SMILES: C#CCN(C(=O)CC(O)(CC(=O)O)C(=O)O)[C@@H]1CCc2ccccc21]

or a salt thereof.

The subject invention also provides a composition comprising a compound having the structure:

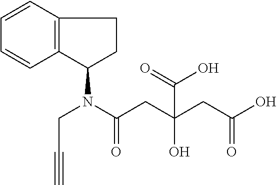 [SMILES: C#CCN(C(=O)CC(O)(CC(=O)O)C(=O)O)[C@@H]1CCc2ccccc21]

or a salt thereof, wherein the composition is free of rasagiline or a salt thereof.

The subject invention further provides a process for preparing rasagiline citramide comprising the steps of:
a) mixing citric acid with thionyl chloride in a first solvent under an inert atmosphere at a temperature of less than 30° C. to obtain trimethyl citrate;
b) mixing trimethyl citrate obtained from step a) and NaOH solution in a second solvent at a temperature of less than 30° C. to obtain 1,2-dimethyl citrate;
c) mixing 1,2-Dimethyl citrate obtained from step b) and thionyl chloride in a third solvent at a temperature of less than 30° C. to obtain a residue oil;
d) mixing the residue oil from step c) and a mixture of rasagilne and triethylamine in the third solvent at a temperature of less than 30° C. to obtain 1-rasagiline-2,3-dimethyl citramide; and
e) mixing an aqueous solution of LiOH and 1-rasagiline-2,3-dimethyl citramide obtained from step d) in a combination of solvents at a temperature of less than 30° C.; and
f) adjusting the pH of the reaction mixture of step e) with an acid to obtain 1-rasagiline citramide.

In an embodiment of the process, in step a) the first solvent is absolute methanol and the inert atmosphere is a nitrogen atmosphere.

In another embodiment of the process, in step b) the second solvent is aqueous methanol.

In yet another embodiment of the process, in steps c) and d) the third solvent is methylene chloride.

In yet another embodiment of the process, in step e) the combination of solvents is dioxane and water.

In yet another embodiment of the process, in step f) the acid is HCl.

The present invention yet further provides a pharmaceutical composition comprising rasagiline or a pharmaceutically acceptable salt thereof, citric acid, rasagiline citramide or a salt thereof, and at least one pharmaceutically acceptable carrier, wherein rasagiline citramide is present in the pharmaceutical composition in an amount greater than about 0.03%, by weight, relative to the amount of rasagiline, based on a determination by an HPLC method, and wherein if the pharmaceutical composition is at least six months old then the temperature of the pharmaceutical composition during such period did not exceed ambient temperature for a total period of four months or more.

The present invention yet further provides a pharmaceutical composition comprising rasagiline or a pharmaceutically acceptable salt thereof, citric acid, rasagiline citramide or a salt thereof, and at least one pharmaceutically acceptable carrier, wherein rasagiline citramide is present in the pharmaceutical composition in an amount greater than about 0.03%, by weight, relative to the amount of rasagiline, based on a determination by an HPLC method, and wherein if the pharmaceutical composition is at least four months old then the temperature of the pharmaceutical composition during such period did not exceed ambient temperature for a total period of four months or more.

The present invention yet further provides a pharmaceutical composition comprising rasagiline or a pharmaceutically acceptable salt thereof, citric acid, rasagiline citramide or a salt thereof, and at least one pharmaceutically acceptable carrier, wherein rasagiline citramide is present in the pharmaceutical composition in an amount greater than about 0.03%, by weight, relative to the amount of rasagiline, based on a determination by an HPLC method, and wherein if the pharmaceutical composition is at least six months old then the temperature of the pharmaceutical composition during such period did not exceed ambient temperature for a total period of six months or more.

In an embodiment of the pharmaceutical composition, the amount of rasagiline citramide is greater than about 0.15, by weight, relative to the amount of rasagiline, based on a determination by en HPLC method.

In another embodiment of the pharmaceutical composition, the rasagiline citramide is present in the pharmaceutical composition in an amount not more than 1.0%, by weight, relative to the amount of rasagiline.

In yet another embodiment of the pharmaceutical composition, the pharmaceutical composition is less than one week old, and the temperature during the less than one week did not exceed ambient temperature.

In yet another embodiment of the pharmaceutical composition, the pharmaceutical composition comprises rasagiline as free base.

In yet another embodiment of the pharmaceutical composition, the pharmaceutical composition comprises the pharmaceutically acceptable salt of rasagiline, and which salt is rasagiline citrate.

In yet another embodiment of the pharmaceutical composition, the pharmaceutical composition is a solid pharmaceutical composition.

In yet another embodiment of the pharmaceutical composition, the pharmaceutical composition is in tablet form.

The subject invention yet further provides a pharmaceutical composition in tablet form comprising a core and a coating, wherein the core of the tablet comprises an amount of rasagiline or a pharmaceutically acceptable salt thereof, citric acid and mannitol, wherein the weight ratio of mannitol to citric acid is between 45 to 1 and 10 to 1, and further comprises rasagiline citramide or a salt thereof such that the rasagiline citramide is present in the pharmaceutical composition in an amount greater than about 0.03%, by weight, relative to the amount of rasagiline, based on a determination by an HPLC method.

In an embodiment of the pharmaceutical composition in tablet form, in the core of the tablet the weight ratio of mannitol to citric acid is between 30 to 1 and 25 to 1.

In another embodiment of the pharmaceutical composition in tablet form, the tablet is less than one week old, and the temperature during the less than one week did not exceed ambient temperature.

In yet another embodiment of the pharmaceutical composition in tablet form, the core of the tablet comprises an amount of rasagiline and citric acid, about 59.91 of mannitol, about 0.53% of aerosil, about 6.6% of starch NE, about 26.3% of pregelatinized starch, about 2.0% of stearic acid, and about 2.0% of talc, by weight, relative to the weight of the core of the tablet.

In yet another embodiment of the pharmaceutical composition in tablet form, the core of the tablet comprises an amount of rasagiline and citric acid, 45.5 mg of mannitol, 0.4 mg of aerosil, 5.0 mg of starch NE, 20.0 mg of pregelatinized starch, 1.5 mg of stearic acid, 1.5 mg of talc, and the coating of the tablet comprises two coating layers, of which the inner of the two coating layers comprises 3.5 mg of hypromellose and the outer of the two coating layers comprises 4.0 mg of methacrylic acid ethyl acrylate copolymer, 0.8 mg of triethyl citrate, and 1.9 mg of talc extra fine.

In yet another embodiment of the pharmaceutical composition in tablet form, the amount of rasagiline in the core is 0.5 mg.

In yet another embodiment of the pharmaceutical composition in tablet form, the core of the tablet comprises an amount of rasagiline and citric acid, about 59.2% of mannitol, about 0.53% of aerosil, about 6.6% of starch NF, about 26.3% of pregelatinized starch, about 2.0% of stearic acid, and about 2.0% of talc, by weight, relative to the weight of the core of the tablet.

In yet another embodiment of the pharmaceutical composition in tablet form, the core of the tablet comprises an amount of rasagiline and citric acid, 45.0 mg of mannitol, 0.4 mg of aerosil, 5.0 mg of starch NE, 20.0 mg of pregelatinized starch, 1.5 mg of stearic acid, 1.5 mg of talc, and the coating of the tablet comprises two coating layers, of which the inner of the two coating layers comprises 3.5 mg of hypromellose and the outer of the two coating layers comprises 4.0 mg of methacrylic acid ethyl acrylate copolymer, 0.8 mg of triethyl citrate, and 1.9 mg of talc extra fine.

In yet another embodiment of the pharmaceutical composition in tablet form, the amount of rasagiline in the core is 1.0 mg.

In yet another embodiment of the pharmaceutical compositions in tablet form, not more than about 1.0% by weight of R(+)-N-methyl-propargyl-aminoindan or a salt thereof is in the pharmaceutical composition relative to the amount of rasagiline.

In yet another embodiment of the pharmaceutical compositions in tablet form, not more than about 1.0% by weight of R(+)-N-formyl-propargyl-aminoindan or a salt thereof is in the pharmaceutical composition relative to the amount of rasagiline.

In yet another embodiment of the pharmaceutical compositions in tablet form, not more than about 0.50% by weight of R(+)-N-formyl-propargyl-aminoindan or a salt thereof is in the pharmaceutical composition relative to the amount of rasagiline.

In yet another embodiment of the pharmaceutical composition in tablet form, the tablet is further coated with a light-resistant coating.

In yet another embodiment of the pharmaceutical composition in tablet form, the light-resistant coating is a coating comprising titanium dioxide.

The invention yet further provides a process for preparing a pharmaceutical composition comprising rasagiline or a pharmaceutically acceptable salt thereof, and at least one pharmaceutically acceptable carrier, comprising:
a) obtaining a batch of rasagiline or a pharmaceutically acceptable salt thereof;
b) determining the amount of rasagiline citramide in the batch using a suitable apparatus; and
c) preparing the pharmaceutical composition from the batch only if the batch is determined to have less than about 1.0% rasagiline citramide by weight relative to the amount of rasagiline.

The invention yet further provides a process for preparing a packaged pharmaceutical composition comprising rasagiline or a pharmaceutically acceptable salt thereof comprising:
a) obtaining a pharmaceutical composition of rasagiline or a pharmaceutically acceptable salt thereof;
b) analyzing the pharmaceutical composition for the presence of rasagiline citramide by a suitable apparatus; and
c) packaging the pharmaceutical composition only if the amount of rasagiline citramide is not more than about 1.0% by weight relative to the amount of rasagiline.

The invention yet further provides a process of distributing a validated batch of a pharmaceutical composition comprising rasagiline or a pharmaceutically acceptable salt thereof and at least one pharmaceutically acceptable carrier, comprising:
a) obtaining a batch of the pharmaceutical composition;
b) performing stability testing with a sample of the batch;
c) determining the total amount of rasagiline citramide in the sample of the batch by a suitable apparatus after stability testing;
d) validating the batch for distribution only if the sample of the batch after stability testing is determined to have not more than about 1.0% by weight of rasagiline citramide relative to the amount of rasagiline; and
e) distributing the validated batch.

In an embodiment of the processes disclosed herein, the pharmaceutical composition comprises rasagiline free base.

In another embodiment of the processes disclosed herein, the pharmaceutical composition comprises rasagiline citrate.

The invention yet further provides rasagiline citramide or a salt thereof for use, as a reference standard to detect trace amounts of rasagiline citramide in a pharmaceutical composition comprising rasagiline or a pharmaceutically acceptable salt of rasagiline.

The invention yet further provides a method for treating Parkinson's disease in a patient comprising administering to the patient an amount of the pharmaceutical compositions disclosed herein effective to treat Parkinson's disease in the patient.

Every embodiment disclosed herein can be combined with every other embodiment of the subject invention, unless specified otherwise.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1, 0.2 . . . 0.9; and 1, 2 . . . 49 mg unit amounts are included as embodiments of this invention.

It will be noted that the structure of the compounds of this invention includes an asymmetric carbon atom and thus the compounds occur as racemates, racemic mixtures, and isolated single enantiomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art or by processes analogous to those described in the Examples disclosed herein using an appropriate isotopically-labeled reagents in place of the non-labeled reagents employed.

A characteristic of a compound refers to any quality that a compound exhibits, e.g., peaks or retention times, as determined by 1H nuclear magnetic spectroscopy, mass spectroscopy, infrared, ultraviolet or fluorescence spectrophotometry, gas chromatography, thin layer chromatography, high performance liquid chromatography, elemental analysis, Ames test, dissolution, stability and any other quality that can be determined by an analytical method. Once the characteristics of a compound are known, the information can be used to, for example, screen or test for the presence of the compound in a sample.

As used herein, a “pharmaceutically acceptable salt” of rasagiline includes citrate, tannate, malate, mesylate, maleate, fumarate, tartrate, esylate, p-toluenesulfonate, benzoate, acetate, phosphate and sulfate salts. For the preparation of pharmaceutically acceptable acid addition salts of the compounds of the invention, the free base can be reacted with the desired acids in the presence of a suitable solvent by conventional methods.

Rasagiline can also be used in its free base form. A process of manufacture of the rasagiline free base is described in U.S. Pat. Nos. 7,750,051 and 7,968,749, the contents of which are hereby incorporated by reference.

As used herein, “drug substance” refers to the active ingredient in a drug product, which provides pharmacological activity or other direct effect in the diagnosis, cure, mitigation, treatment, or prevention of disease, or to affect the structure or any function of the body of man or animals.

As used herein, “drug product” refers to the finished dosage form containing the drug substance as well as at least one pharmaceutically acceptable carrier.

As used herein, an “isolated” compound is a compound isolated from the crude reaction mixture following an affirmative act of isolation. The act of isolation necessarily involves separating the compound from the other known components of the crude reaction mixture, with some impurities, unknown side products and residual amounts of the other known components of the crude reaction mixture permitted to remain. Purification is an example of an affirmative act of isolation.

As used herein, a composition that is “free” of a chemical entity means that the composition contains, if at all, an amount of the chemical entity which cannot be avoided following an affirmative act intended to purify the composition by separating the chemical entity from the composition. A composition which is “free” of rasagiline of a salt thereof, if present, as used herein, means that the rasagiline or a salt thereof is a minority component relative to the amount of rasagiline citramide, by weight.

As used herein, “stability testing” refers to tests conducted at specific time intervals and various environmental conditions (e.g., temperature and humidity) to see if and to what extent a drug product degrades over its designated shelf life time. The specific conditions and time of the tests are such that they accelerate the conditions the drug product is expected to encounter over its shelf life. For example, detailed requirements of stability testing for finished pharmaceuticals are codified in 21 C.F.R §211.166, the entire content of which is hereby incorporated by reference.

As used herein, a pharmaceutical composition which is “X weeks old” refers to the period of time, e.g. one week old or four months old, since the pharmaceutical composition was made.

As used herein, “ambient temperature” refers to a temperature of from about 20° C. to about 30° C.

A “detection limit” for an analytical method used in screening or testing for the presence of a compound in a sample is a threshold under which the compound in a sample cannot be detected by the analytical method, e.g. an HPLC, PS, NMR, or FT-IR method.

As used herein, “about” in the context of a measurable numerical value means the numerical value within the standard error of the analytical method used to measure.

A dosage unit may comprise a single compound or mixtures of compounds thereof. A dosage unit can be prepared for oral dosage forms, such as tablets, capsules, pills, powders, and granules.

As used herein, a “pharmaceutically acceptable” carrier or excipient is one that is suitable for use with humans and/or animals without undue adverse side effects (such as toxicity, irritation, and allergic response) commensurate with a reasonable benefit/risk ratio.

Specific examples of pharmaceutical acceptable carriers and excipients that may be used to formulate oral dosage forms are described, e.g., in U.S. Pat. No. 6,126,968 to Peskin et al., issued Oct. 3, 2000. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, melting agents, stabilizing agents, solubilizing agents, antioxidants, buffering agent, chelating agents, fillers and plasticizers. For instance, for oral administration in the dosage unit form of a tablet or capsule, the active drug component can be combined with an oral, non-toxic, pharmaceutically acceptable, inert carrier such as gelatin, agar, starch, methyl cellulose, dicalcium phosphate, calcium sulfate, mannitol, sorbitol, microcrystalline cellulose and the like. Suitable binders include starch, gelatin, natural sugars such as corn starch, natural and synthetic gums such as acacia, tragacanth, or sodium alginate, povidone, carboxymethylcellulose, polyethylene glycol, waxes, and the like. Antioxidants include ascorbic acid, fumaric acid, citric acid, malic acid, gallic acid and its salts and esters, butylated hydroxyanisole, editic acid. Lubricants used in these dosage forms include sodium oleate, sodium stearate, sodium benzoate, sodium acetate, stearic acid, sodium stearyl fumarate, talc and the like. Disintegrators include, without limitation, starch, methyl cellulose, agar, bentonite, xanthan gum, croscarmellose sodium, sodium starch glycolate and the like, suitable plasticizers include triacetin, triethyl citrate, dibutyl sebacate, polyethylene glycol and the like.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Preparation of Rasagiline Citramide

1. Introduction

Stability study of rasagiline has drug product reveals a new impurity that was detected as early as 1 month and quantifiable thereafter and after 4 and 6 months of storage at 40° C./75% RH. This impurity is confirmed to be product-related and is not affected by the analytical equipment or method. The impurity is assumed to be depending on temperature and is formed only when both Rasagiline and citric acid are presented in the product. The data from MS analysis matched with a structure containing both citric acid and Rasagiline base (M/Z=345).

Molecular Formula and Structure of Rasagiline Citramide

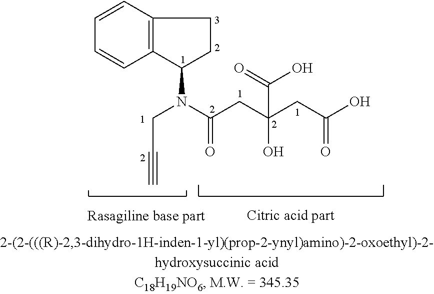 [SMILES: C#CCN(C(=O)CC(O)(CC(=O)O)C(=O)O)[C@@H]1CCc2ccccc21]

2. Isolation of Rasagiline Citramide Impurity

Rasagiline citramide was isolated by employing similar conditions as in the stability study (where it was originally found) but on a larger scale so that enough material would be formed and subsequently isolated for characterization.

Citric acid (60.0 g), rasagiline base (30.0 g) and water (10 ml) were heated under argon at 80° C. for 24 hr. The resulting oil was dissolved in water (300 ml) and 1N HCl (pH=1-2) was added. The reaction mixture was extracted by ethyl acetate (4×100 ml), the ethyl acetate extract was dried over sodium sulfate and evaporated to dryness. The resulting green oily residue (1.0 g) was purified by column chromatography (2% methanol in DCM) to give 0.22 g of a yellow solid.

The yellow solid obtained was characterized by 700 MHz ¹H & ¹³C-NMR and MS to be Rasagiline citramide having the structure:

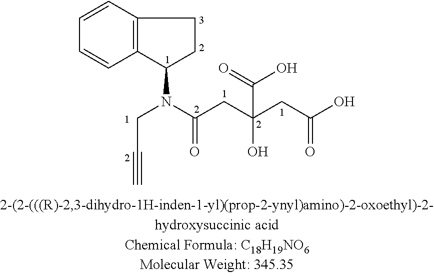 [SMILES: C#CCN(C(=O)CC(O)(CC(=O)O)C(=O)O)[C@@H]1CCc2ccccc21]

Due to the fact that the amidation took place on the 1-carboxlic acid, the prochiral center on carbon 2 became chiral and the Rasagiline citramide obtained as a diastereomeric mixture as shown below:

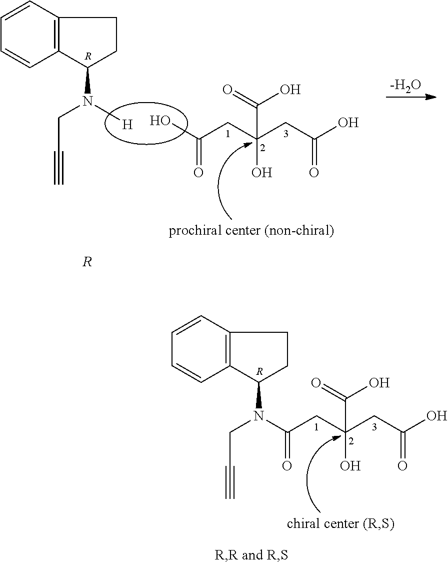

The NMR spectra are complicated due to the diastereomeric mixture, and they are even more complicated due to the two rotamers that are formed around the amid bond as shown below:

Rotamers Mixture of Rasagiline Citramide

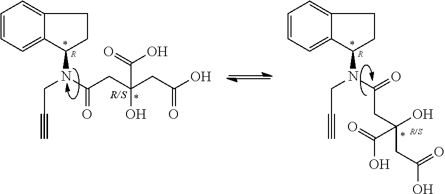

3. Synthesis of Rasagiline Citramide

The synthesis of Rasagiline citramide is described in the Scheme below. In the first step, citric acid is esterified to trimethyl citrate. In the second step trimethyl citrate is converted to 1,2-dimethyl citrate by a selective sterically controlled saponification. The third step is an amidation reaction between Rasagiline and 1,2-dimethyl citramide starting with activating the free carboxyl to the acyl chloride derivative followed by the addition of Rasagiline base. In the last step the esters are hydrolyzed and Rasagiline citramide is obtained. The overall yield is 3.5%.

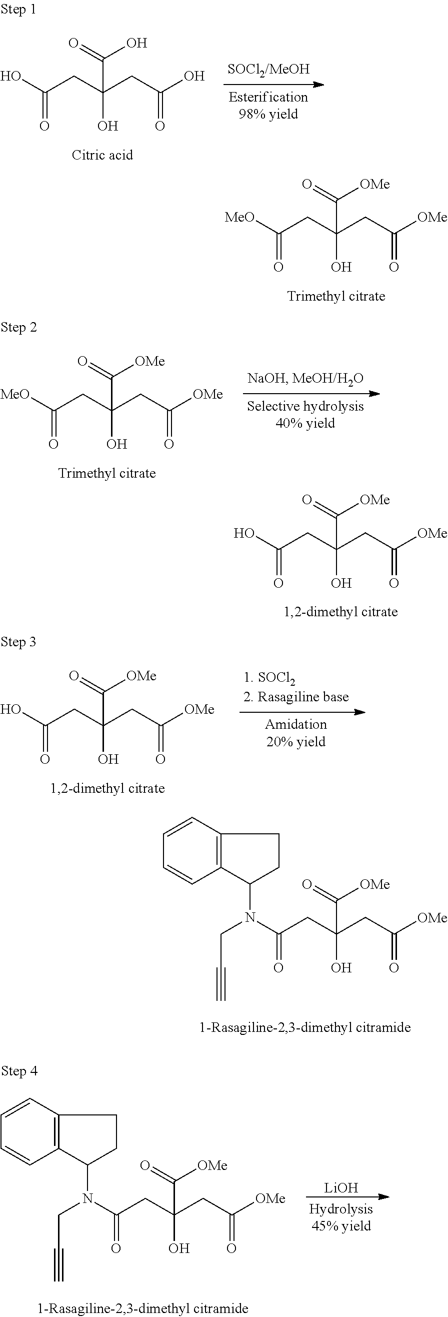

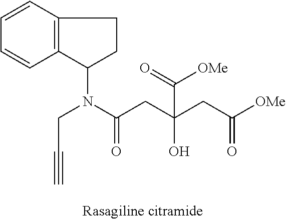 [SMILES: C#CCN(C(=O)CC(O)(CC(=O)OC)C(=O)OC)C1CCc2ccccc21]

### Step 1. Preparation of Trimethyl Citrate

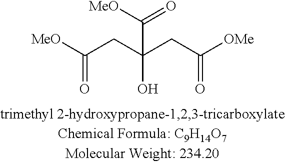 [SMILES: COC(=O)CC(O)(CC(=O)OC)C(=O)OC]

To a stirred solution of citric acid (9.00 g, 46.8 mmol) in absolute methanol at 0° C. under a nitrogen atmosphere, thionyl chloride (20.50 mL, 0.28 mol, 2 eq.) was carefully added. The reaction mixture was stirred at 0° C. for an hour then at room temperature overnight. The volatiles were then removed in vacuum. The residual solid was recrystallized from hexane/ethyl acetate to yield 10.809 (98%) of the title compound as white crystals.

### Step 2. Preparation of 1,2-dimethyl Citrate

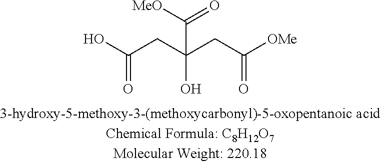 [SMILES: COC(=O)CC(O)(CC(=O)O)C(=O)OC]

NaOH (0.1N, 215 ml) was added to a solution of trimethyl citrate (10 g, 42.7 mmol) in 50% aqueous MeOH (200 ml) over 2 h with vigorous stirring at RT. The solution was concentrated to about 150 ml and extracted with ethyl acetate (3×150 ml). The aqueous phase was acidified with 1N HCl (45 ml) and extracted with ethyl acetate (3×150 ml). The combined organic phases were dried (MgSO₄) and concentrated to provide 3.7 g (39%) of the product as a colorless oil.

### 3. Preparation of 1-Rasagiline-2,3-dimethyl Citramide

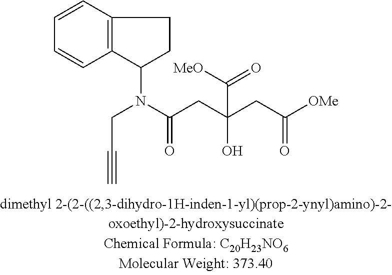 [SMILES: C#CCN(C(=O)CC(O)(CC(=O)OC)C(=O)OC)C1CCc2ccccc21]

1,2-Dimethyl citrate (5.4 g, 24.5 mmol) was dissolved in DCM (100 ml) and thionyl chloride (3.8 g, 32 mmol) was carefully added at RT. The resulted clear solution was stirred for 2 hr at RT and evaporated to dryness. The colorless residue was dissolved in DCM (50 ml) and was then added to a mixture of PAI (8.4 g, 50 mmol) and triethylamine (3.7 g, 37 mmol) in DCM (100 ml) for 15 min at 0-5° C. followed by stirring for 0.5 hr at 0-5° C. and 2 hr at R.T. The reaction mixture was then washed with 1N HCl and water, the organic solution was dried over sodium sulfate and evaporated to dryness to yield 2.9 g of green solid that was purified by column chromatography (20% ethyl acetate in hexane). 1.8 g (20%) of colorless solid was obtained.

### Step 4. 1-Rasagiline Citramide

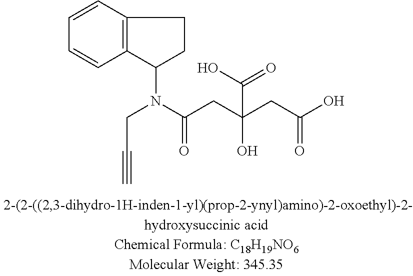 [SMILES: C#CCN(C(=O)CC(O)(CC(=O)O)C(=O)O)C1CCc2ccccc21]

A solution of LiOH (2.3 g, 60 mmol) in water (30 ml) was dropwise added into a solution of 1-Rasagiline-2,3-dimethyl citramide (5.6 g, 15.0 mmol) in dioxane (100 ml) and water (10 ml) at 0-5° C. for 0.5 hr. The reaction mixture was stirred for another 0.5 hr at 0-5° C. and 1.5 hr at RT, evaporated to reduce volume up to 20-30% and diluted with water (100 ml). The reaction mixture was acidified by HCl (37%) to pH 1-2 and extracted with ethyl acetate (3×100 ml). The organic phase was dried over sodium sulfate and evaporated to dryness to gibe 4.2 g of colorless oil that was purified by column chromatography (2% methanol in DCM) to yield 2.3 g (45%) of a white solid.

Purified rasagiline citramide from the above experiments was characterized by Element Analysis, ¹H-NMR, ¹³C-NMR and MS.

Elemental Analysis

The analysis for C. H and N was performed using a Perkin-Elmer 2400 series II analyzer.

TABLE 1
Element Analysis Results for Rasagiline Citramide

| Element | | % C | % H | % N |
|---|---|---|---|---|
| Theoretical | | 62.60 | 5.55 | 4.06 |
| Experimental | Sample 1 | 60.85 | 5.49 | 3.70 |
| | Sample 2 | 60.81 | 5.52 | 3.70 |

The results of the elemental analysis correspond to the molecular formula of rasagiline citramide.

NMR Spectroscopy

The ¹H-NMR and ¹³C-NMR spectra of rasagiline citramide were recorded on a Broker Avance III 700 instrument at 700 and 176 MHz respectively. The spectra were run at room temperature (T=300K) in D₆-DMSO as a solvent with TMS as internal reference. The shift assignments are summarized in Table 2 with the designations shown in structure below. The spectra are consistent with the expected structure.

Structure of Rasagiline Citramide with Designations Used for the Attribution of ¹³C and ¹H-NMR Shifts

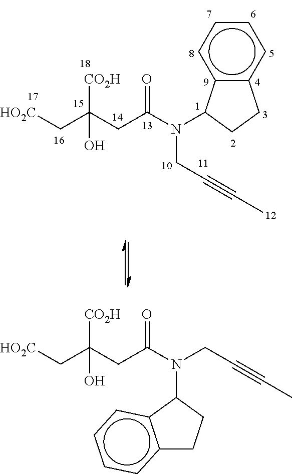

TABLE 2
¹H-NMR and ¹³C-NMR Chemical Shifts of Rasagiline
Citramide in D₆-DMSO

| | Rotamer “A” | | Rotamer “B” | |
|---|---|---|---|---|
| | ¹H | ¹³C | ¹H | ¹³C |
| 1 | 6.01, 6.04 | 58.46, 58.54 | 5.61, 5.63 | 62.46, 62.50 |
| 2 | ca. 2.05 | 28.88, 28.92 | 2.18 | 29.83, 29.98 |
| | | | 2.43 | |
| 3 | 2.82 | 29.63 | 2.82 | 29.45, 29.47 |
| | 2.98 | | 2.98 | |
| 4 | — | 143.43 | — | 143.10 |
| 5-8 | 7.0-7.3 | 123-128 | 7.0-7.3 | 123-128 |
| 9 | — | 140.64, | — | 140.22, |
| | | 140.68 | | 140.26 |
| 10 | 3.55, 3.61 | 32.95 | 3.35 | 30.77 |
| | 4.09, 4.14 | | 4.02, 4.05 | |
| 11 | — | 80.67, 80.70 | — | 81.23, 81.30 |
| 12 | 3.28, 3.30 | 74.30, 74.37 | 2.96 | 72.21, 72.26 |
| 13 | — | 170.32, | — | 169.58, |
| | | 170.64 | | 169.89 |
| 14 | 2.89, 3.00 | 40.70, 41.25 | 2.8-3.2 | 40.25, 40.61 |
| | 3.16, 3.23 | | | |
| 15 | — | 73.08, 73.15 | — | 73.01, 73.04 |
| 16 | 2.65-2.80 | 42.49, 43.16 | 2.65-2.8 | 42.55, 42.77 |
| 17 | — | 174.63 | — | 174.56, |
| | | | | 174.61 |
| 18 | — | 171.16, | — | 171.23, |
| | | 171.30 | | 171.29 |

¹brs = broad singlet; brm = broad multiplet

Mass Spectroscopy (MS)

The mass spectrum of rasagiline citramide was performed on a TOF-MS-ES instrument. The spectrum exhibits quasi-molecular ions at m/z 346 [M+H], which is in agreement with the molecular formula of rasagiline citramide.

### Example 2

### Stability Study of Rasagiline Base Delayed Release Tablets:

Rasagiline base delayed release tablets were subject to stability testing under various storage conditions. The tablets were prepared according to procedures described in Examples 3b and 3d of United States Application Publication No. 2010/0189787.

The tablets were analyzed for the presence of rasagiline citramide after storage under various conditions as summarized below.

TABLE 4
Percentage of Rasagiline Citramide Present at
40 ± 2° C./75 ± 5% RH in 1 mg Rasagiline Base EC Tablets

| | | Rasagiline citramide (%) | | |
|---|---|---|---|---|
| | | Aluminum | | HDPE 100 cc |
| | | Silver/Aluminum | HDPE 100 cc | bottles with |
| | | Soft blister | bottles/CR PP | CR PP |
| Batch | Timepoint | cards, 10 | Induction cap, | Induction cap, |
| Number | (months) | tablets | 90 tablets | 28 tablets |
| A | 0 | <0.03 (DL) | <0.03 (DL) | <0.03 (DL) |
| | 1 | <0.1 (QL) | <0.1 (QL) | <0.1 (QL) |
| | 2 | 0.2 | 0.3 | 0.3 |
| | 3 | 0.4 | 0.4 | 0.4 |
| | 6 | 0.6 | 0.7 | 0.8 |
| B | 0 | <0.03 (DL) | <0.03 (DL) | <0.03 (DL) |
| | 1 | <0.1 (QL) | <0.1 (QL) | <0.1 (QL) |
| | 2 | 0.2 | 0.2 | 0.3 |
| | 3 | 0.4 | 0.4 | 0.4 |
| | 6 | 0.6 | 0.7 | 0.8 |
| C | 0 | <0.03 (DL) | <0.03 (DL) | <0.03 (DL) |
| | 1 | <0.1 (QL) | <0.1 (QL) | <0.1 (QL) |
| | 2 | 0.2 | 0.3 | 0.3 |
| | 3 | 0.4 | 0.4 | 0.5 |
| | 6 | 0.6 | 0.7 | 0.8 |

TABLE 5
Percentage of Rasagiline Citramide Present at
25 ± 2° C./60 ± 5% RH in 1 mg Rasagiline Base EC Tablets

| | | Rasagiline citramide (%) | | |
|---|---|---|---|---|
| | | Aluminum | | HDPE 100 cc |
| | | Silver/Aluminum | HDPE 100 cc | bottles |
| | | Soft blister | bottles/CR PP | with CR PP |
| Batch | Timepoint | cards, 10 | Induction cap, | Induction cap, |
| Number | (months) | tablets | 90 tablets | 28 tablets |
| A | 0 | <0.03 (DL) | <0.03 (DL) | <0.03 (DL) |
| | 3 | <0.04 (DL) | <0.04 (DL) | <0.04 (DL) |
| | 6 | <0.1 (QL) | <0.1 (QL) | <0.1 (QL) |
| | 9 | <0.1 (QL) | <0.1 (QL) | <0.1 (QL) |
| | 12 | <0.1 | <0.1 | 0.1 |
| B | 0 | <0.03 (DL) | <0.03 (DL) | <0.03 (DL) |
| | 3 | <0.04 (DL) | <0.04 (DL) | <0.04 (DL) |
| | 6 | <0.1 (QL) | <0.1 (QL) | <0.1 (QL) |
| | 9 | <0.1 (QL) | <0.1 (QL) | <0.1 (QL) |
| | 12 | <0.1 (QL) | <0.1 | 0.1 |
| C | 0 | <0.03 (DL) | <0.03 (DL) | <0.03 (DL) |
| | 3 | <0.04 (DL) | <0.04 (DL) | <0.04 (DL) |
| | 6 | <0.1 (QL) | <0.1 (QL) | <0.1 (QL) |
| | 9 | <0.1 (QL) | <0.1 (QL) | <0.1 (QL) |
| | 12 | <0.1 (QL) | <0.1 | <0.1 |

TABLE 6
Percentage of Rasagiline Citramide Present at
40 ± 2° C./75 ± 5% RH in 1 mg Rasagiline Base EC Tablets

| | | Rasagiline citramide (%) | | |
|---|---|---|---|---|
| | | Aluminum | | HDPE 100 cc |
| | | Silver/Aluminum | HDPE 100 cc | bottles |
| | | Soft blister | bottles/CR PP | with CR PP |
| Batch | Timepoint | cards, 10 | Induction cap, | Induction cap, |
| Number | (months) | tablets | 90 tablets | 28 tablets |
| D | 0 | <0.03 (DL) | <0.03 (DL) | <0.03 (DL) |
| | 1 | <0.03 (DL) | <0.03 (DL) | <0.1 (QL) |
| | 2 | 0.1 | 0.1 | 0.1 |
| | 3 | 0.2 | 0.2 | 0.2 |
| | 6 | 0.3 | 0.3 | 0.4 |
| E | 0 | <0.03 (DL) | <0.03 (DL) | <0.03 (DL) |
| | 1 | <0.1 (QL) | <0.1 (QL) | <0.03 (DL) |
| | 2 | 0.1 | 0.1 | 0.1 |
| | 3 | 0.2 | 0.2 | 0.2 |
| | 6 | 0.3 | 0.3 | 0.4 |
| F | 0 | <0.03 (DL) | <0.03 (DL) | <0.03 (DL) |
| | 1 | <0.1 (QL) | <0.1 (QL) | <0.03 (DL) |
| | 2 | 0.1 | 0.1 | 0.1 |
| | 3 | 0.2 | 0.2 | 0.2 |
| | 6 | 0.3 | 0.3 | 0.3 |
| G | 0 | <0.03 (DL) | <0.03 (DL) | <0.03 (DL) |
| | 1 | <0.03 (DL) | <0.1 (QL) | <0.1 (QL) |
| | 2 | 0.1 | 0.1 | 0.1 |
| | 3 | 0.2 | 0.2 | 0.2 |
| | 6 | 0.3 | 0.4 | 0.4 |

TABLE 7
Percentage of Rasagiline Citramide Present at
25 ± 2° C./60 ± 5% RH in 1 mg Rasagiline Base EC Tablets

| | | Rasagiline citramide (%) | | |
|---|---|---|---|---|
| | | Aluminum | | HDPE 100 cc |
| | | Silver/Aluminum | HDPE 100 cc | bottles |
| | | Soft blister | bottles/CR PP | with CR PP |
| Batch | Timepoint | cards, 10 | Induction cap, | Induction cap, |
| Number | (months) | tablets | 90 tablets | 28 tablets |
| D | 0 | <0.03 (DL) | <0.03 (DL) | <0.03 (DL) |
| | 3 | <0.04 (DL) | <0.04 (DL) | <0.04 (DL) |
| | 6 | <0.03 (DL) | <0.03 (DL) | <0.03 (DL) |
| | 9 | <0.1 (QL) | <0.1 (QL) | <0.1 (QL) |
| | 12 | <0.1 | <0.1 | <0.1 |
| E | 0 | <0.03 (DL) | <0.03 (DL) | <0.03 (DL) |
| | 3 | <0.04 (DL) | <0.04 (DL) | <0.04 (DL) |
| | 6 | <0.03 (DL) | <0.03 (DL) | <0.03 (DL) |
| | 9 | <0.1 (QL) | <0.1 (QL) | <0.1 (QL) |
| | 12 | <0.1 | <0.1 | <0.1 |
| F | 0 | <0.03 (DL) | <0.03 (DL) | <0.03 (DL) |
| | 3 | <0.04 (DL) | <0.04 (DL) | <0.04 (DL) |
| | 6 | <0.03 (DL) | <0.03 (DL) | <0.03 (DL) |
| | 9 | <0.1 (QL) | <0.1 (QL) | <0.1 (QL) |
| | 12 | <0.1 | <0.1 | <0.1 |
| G | 0 | <0.03 (DL) | <0.03 (DL) | <0.03 (DL) |
| | 3 | <0.04 (DL) | <0.04 (DL) | <0.04 (DL) |
| | 6 | <0.03 (DL) | <0.03 (DL) | <0.03 (DL) |
| | 9 | <0.1 (QL) | <0.1 (QL) | <0.1 (QL) |
| | 12 | <0.1 | <0.1 | <0.1 |

Results and Discussion:

The testing results demonstrate that rasagiline drug product prepared under standard manufacturing conditions exhibits non-detectable level of rasagiline citramide.

The testing results also demonstrates that rasagiline citramide impurity may form during storage of rasagiline drug product under accelerated conditions, e.g. at 60±5% and 75±5% RH, and at 25±2° and 40±2° C., as shown in Tables 4-7.

1. An isolated compound having the structure: 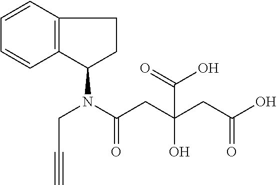 [SMILES: C#CCN(C(=O)CC(O)(CC(=O)O)C(=O)O)[C@@H]1CCc2ccccc21]
or a salt thereof.

2. A composition comprising a compound having the structure: 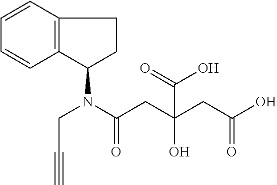 [SMILES: C#CCN(C(=O)CC(O)(CC(=O)O)C(=O)O)[C@@H]1CCc2ccccc21]
or a salt thereof,
wherein the composition is free of rasagiline or a salt thereof.

3. A process for preparing rasagiline citramide recited in claim 1 comprising the steps of:
a) mixing citric acid with thionyl chloride in a first solvent under an inert atmosphere at a temperature or less than 30° C. to obtain trimethyl citrate;
b) mixing trimethyl citrate obtained from step a) and NaOH solution in a second solvent at a temperature of less than 30° C. to obtain 1,2-dimethyl citrate;
c) mixing 1,2-Dimethyl citrate obtained from step b) and thionyl chloride in a third solvent at a temperature of less than 30° C. to obtain a residue oil;
d) mixing the residue oil from step c) and a mixture of rasagilne and triethylamine in the third solvent at a temperature of less than 30° C. to obtain 1-rasagiline-2,3-dimethyl citramide; and
e) nixing an aqueous solution of LiOH and 1-rasagiline-2,3-dimethyl citramide obtained from step d) in a combination of solvents at a temperature of less than 30° C.; and
f) adjusting the pH of the reaction mixture of step e) with an acid to obtain 1-rasagiline citramide.

4-8. (canceled)

9. A pharmaceutical composition comprising rasagiline or a pharmaceutically acceptable salt thereof, citric acid, rasagiline citramide or a salt thereof, and at least one pharmaceutically acceptable carrier,
wherein rasagiline citramide is present in the pharmaceutical composition in an amount greater than about 0.03%, by weight, relative to the amount of rasagiline, based on a determination by an HPLC method,
and
wherein if the pharmaceutical composition is at least six months old then the temperature of the pharmaceutical composition during such period did not exceed ambient temperature for a total period of four months or more.

10. The pharmaceutical composition of claim 9, wherein the amount of rasagiline citramide is greater than about 0.1%, by weight, relative to the amount of rasagiline, based on a determination by an HPLC method.

11. The pharmaceutical composition of claim 9, wherein the rasagiline citramide is present in the pharmaceutical composition in an amount not more than 1.0%, by weight, relative to the amount of rasagiline.

12. The pharmaceutical composition of claim 9, which is less than one week old, and the temperature during the less than one week did not exceed ambient temperature.

13. The pharmaceutical composition of claim 9, which comprises rasagiline as free base.

14. The pharmaceutical composition of claim 9, which comprises the pharmaceutically acceptable salt of rasagiline, and which salt is rasagiline citrate.

15. The pharmaceutical composition of claim 9, wherein the pharmaceutical composition is a solid pharmaceutical composition.

16. The pharmaceutical composition of claim 15, which is in tablet form.

17. A pharmaceutical composition in tablet form comprising a core and a coating, wherein the core of the tablet comprises an amount of rasagiline or a pharmaceutically acceptable salt thereof, citric acid and mannitol, wherein the weight ratio of mannitol to citric acid is between 45 to 1 and 10 to 1, and further comprises rasagiline citramide or a salt thereof such that the rasagiline citramide is present in the pharmaceutical composition in an amount greater than about 0.03%, by weight, relative to the amount of rasagiline, based on a determination by an HPLC method.

18. The pharmaceutical composition of claim 17 wherein in the core of the tablet the weight ratio of mannitol to citric acid is between 30 to 1 and 25 to 1.

19. The pharmaceutical composition of claim 17 or 18, which is less than one week old, and the temperature during the less than one week did not exceed ambient temperature.

20. The pharmaceutical composition of claim 17, wherein the core of the tablet comprises an amount of rasagiline and citric acid, about 59.9% of mannitol, about 0.53% of aerosil, about 6.6% of starch NF, about 26.3% of pregelatinized starch, about 2.0% of stearic acid, and about 2.0% of talc, by weight, relative to the weight of the core of the tablet.

21. The pharmaceutical composition of claim 20, wherein the core of the tablet comprises an amount of rasagiline and citric acid, 45.5 mg of mannitol, 0.4 mg of aerosil, 5.0 mg of starch NF, 20.0 mg of pregelatinized starch, 1.5 mg of stearic acid, 1.5 mg of talc, and the coating of the tablet comprises two coating of which the inner of the two coating layers comprises 3.5 mg of hypromellose and the cuter of the two coating layers comprises 4.0 mg of methacrylic acid ethyl acrylate copolymer, 0.8 mg of triethyl citrate, and 1.9 mg of talc extra fine.

22. The pharmaceutical composition of claim 17, wherein the amount of rasagiline in the core is 0.5 mg.

23. The pharmaceutical composition of claim 17, wherein the core of the tablet comprises an amount of rasagiline and citric acid, about 59.2% of mannitol, about 0.53% of aerosil, about 6.6% of starch NF, about 26.3% of pregelatinized starch, about 2.0% of stearic acid, and about 2.0% of talc, by weight, relative to the weight of the core of the tablet.

24. The pharmaceutical composition of claim 23, wherein the core of the tablet comprises an amount of rasagiline and citric acid, 45.0 mg of mannitol, 0.4 mg of aerosil, 5.0 mg of starch NE, 20.0 mg of pregelatinized starch, 1.5 mg of stearic acid, 1.5 mg of talc, and the coating of the tablet comprises two coating layers, of which the inner of the two coating layers comprises 3.5 mg of hypromellose and the outer of the two coating layers comprises 4.0 mg of methacrylic acid ethyl acrylate copolymer, 0.8 mg of triethyl citrate, and 1.9 mg of talc extra fine.

25. The pharmaceutical composition of claim 17, wherein the amount of rasagiline in the core is 1.0 mg.

26. The pharmaceutical composition of claim 17, which is less than one week old, and the temperature during the less than one week did not exceed ambient temperature.

27. The pharmaceutical composition of claim 9, wherein not more than about 1.0% by weight of R(+)-N-methyl-propargyl-aminoindan or a salt thereof is in the pharmaceutical composition relative to the amount of rasagiline, or wherein not more than about 1.0% by weight of R(+)-N-formyl-propargyl-aminoindan or a salt thereof is in the pharmaceutical composition relative to the amount of rasagiline.

28-29. (canceled)

30. The pharmaceutical composition of claim 9, wherein the tablet is further coated with a light-resistant coating.

31. (canceled)

32. A process for preparing a pharmaceutical composition comprising rasagiline or a pharmaceutically acceptable salt thereof, and at least one pharmaceutically acceptable carrier, comprising:
a) obtaining a batch of rasagiline or a pharmaceutically acceptable salt thereof;
b) determining the amount of the compound recited in claim 1 in the batch using a suitable apparatus; and c) preparing the pharmaceutical composition from the batch only if the batch is determined to have less than about 1.0% of the compound by weight relative to the amount of rasagiline.

33. A process for preparing a packaged pharmaceutical composition comprising rasagiline or a pharmaceutically acceptable salt thereof comprising:
a) obtaining a pharmaceutical composition of rasagiline or a pharmaceutically acceptable salt thereof;
b) analyzing the pharmaceutical composition for the presence of the compound recited in claim 1 by a suitable apparatus; and
c) packaging the pharmaceutical composition only if the amount of the compound is not more than about 1.0% by weight relative to the amount of rasagiline.

34. A process of distributing a validated batch of a pharmaceutical composition comprising rasagiline or a pharmaceutically acceptable salt thereof and at least one pharmaceutically acceptable carrier, comprising:
a) obtaining a batch of the pharmaceutical composition;
b) performing stability testing with a sample of the batch;
c) determining the total amount of the compound recited in claim 1 in the sample of the batch by a suitable apparatus after stability testing;
d) validating the batch for distribution only if the sample of the batch after stability testing is determined to have not more than about 1.0% by weight of the compound relative to the amount of rasagiline; and
e) distributing the validated batch.

35-37. (canceled)

38. A method for treating Parkinson's disease in a patient comprising administering to the patient an amount of the pharmaceutical composition of claim 9 effective to treat Parkinson's disease in the patient.
